(19) United States
(12) Patent Application Publication
(10) Pub. No.: US 2024/0050502 A1
(43) Pub. Date: Feb. 15, 2024

(54) AMANITA MUSCARIA COMPOUNDS

(71) Applicant: CAAMTECH, INC., Issaquah, WA (US)

(72) Inventor: Andrew R. CHADEAYNE, Issaquah, WA (US)

(21) Appl. No.: 18/257,639

(22) PCT Filed: Dec. 14, 2021
(86) PCT No.: PCT/US2021/063198

§ 371 Date: Jun. 15, 2023

Related U.S. Application Data
(60) Provisional application No. 63/126,019, filed on Dec. 16, 2020.

(51) Int. Cl. A61K 36/07; A61K 45/06; A61K 31/42

(52) U.S. Cl. CPC A61K 36/07; A61K 45/06; A61K 31/42

(57) ABSTRACT

Disclosed are compositions containing two purified Amanita muscaria compounds chosen from the following: ibotenic acid, muscimol, muscazone, stizolobic acid, stizolobinic acid, muscarine, muscamfin, muscaflavin, betalamic acid, muscapurpurin, muscaaurins, acetylcholine, atropine, hyoscyamine, L-3,4-dihydroxyphenylalanine, phallolysin, scopolamine, fucomannogalactan, B(1→6)-branched (1→3)-β-d-glucan, amatoxins, phallotoxins, or the salts of these Amanita muscaria compounds. The disclosure also relates to formulations, including pharmaceutical formulations, of such a composition and an excipient. Pharmaceutical formulations further containing a therapeutically effective amount of a serotonergic drug, a purified psilocybin derivative, a purified cannabinoid, or a purified terpene a disclosed. Also disclosed are methods of regulating the activity of a neurotransmitter receptor and methods of treating a psychological disorder, a compulsive disorder, or a depressive disorder.

### CROSS-REFERENCE TO RELATED APPLICATIONS

This application claims priority to U.S. Provisional Application No. 63/126,019, filed on Dec. 16, 2020, the disclosure of which is incorporated by reference.

### TECHNICAL FIELD

The disclosure relates to new compositions and formulations of purified Amanita muscaria compounds as well as methods for the therapeutic use of those compositions and formulations.

### BACKGROUND

The primary psychotropic compounds in Amanita muscaria are muscimol and ibotenic acid. 1-4 They are derivatives of the neurotransmitters gamma-aminobutyric acid (GABA) and glutamic acid, respectively. As such, these two compounds activate the same receptors as the endogenous compounds would.

One of the first practical uses of A. muscaria was as an insecticide (the word musca comes from the Latin for fly). This application resulted in the other names it is known by, fly agaric and fly amantia.⁵

The minimum dose of ibotenic acid needed to obtain psychedelic effects is 30-60 mg, while central nervous system intoxication with muscimol occurs at 6.0 mg. 6 One fruit body of A. muscaria weighing 50-70 g may contain up to 70 mg of ibotenic acid.

The LD 50 for muscimol intravenous administration in mice is 5.62 mg/kg and 15 mg/kg for ibotenic acid.⁷ The oral LD 50 for ibotenic acid in mice is 38 mg/kg. Based on a 2013 literature review, Stebelska stated that ibotenic acid is “less active but more dangerous” than muscimol.⁶

In a 2008 review, Merova et al. stated that although A. muscaria contains poisonous compounds, the intoxication/psychedelic effects it brings on are rarely lethal.⁸ However, when accidentally ingested, identification of the species is essential so medical treatment can begin as soon as possible. Interestingly, in a review of Amanita poisonings in his book “Toxic Fungi of Western North America,” Duffy observed, “Amanita muscaria has had a higher fatality rate than Amanita pantherina in Germany. In the United States, more deaths have been reported from Amanita pantherina than from Amanita muscaria, which almost never has a fatal outcome here.”

In adults, the ingestion of A. muscaria initially results in symptoms of muscarine poisoning including nausea, vomiting, diarrhea, sweating, vasodilation, and salivation (summarized in Stebelska, 2013 and Michelot and Melendez-Howell, 2003). About 30 minutes after ingestion, the symptoms resemble those of the drug and naturally occurring compound atropine. The symptoms include pupil dilation, dry mouth, elevated body temperature, slowed heart rate, drowsiness, dizziness, hypersensitivity to light, euphoria, motor hyperactivity, delirium, and hallucinations (interestingly, some people report a mystical-type experience). The symptoms intensify over the next 2-3 hours and start declining after about 8 hours. In some fatal cases, the final symptoms may be similar to a stroke.⁹

The symptoms of A. muscaria poisoning follow a more excitatory course in children than adults.¹⁰ These symptoms include myoclonic jerking, muscle fasciculation, and minor to severe seizures.

Muscimol (aka pantherine, agarin) is a potent agonist of GABA A receptors (GABA A R) in the brain. 1142 It is likely the principal psychoactive constituent of A. muscaria. Unlike endogenous GABA, muscimol can cross the blood-brain barrier. Researchers theorize this movement may utilize an active transport mechanism^{9,13,14} or the high lipid membrane solubility of muscimol (or a metabolite/derivative thereof).¹⁵

Activation of GABA A R causes an inhibitory effect, meaning it reduces the transmission of neuronal signals. This inhibition is one reason that GABA receptors and their transport mechanisms are used as targets for drug development.¹⁶ Studies by Ebert et al. in 1997 examined the binding of muscimol to the GABA A Rs with different receptor subunit combinations. The data indicated Ki values from 3.3 to 9.0 nM.¹¹

The GABA A R is a ligand-gated ion channel that modulates chloride ions and, to some extent, bicarbonate ions.¹⁷ The GABA_{A}R has several distinct allosteric binding sites.^{18,19} Two examples of drugs that utilize this are benzodiazepines and barbiturates, designed to target allosteric sites on GABA A R.

Besides its inhibitory receptor activity, muscimol also inhibits the uptake of GABA by neurons and astrocytes.⁶ It is also a substrate for the enzyme that inactivates GABA, GABA-metabolizing enzyme.

Ibotenic acid (aka ibotenate) is a secondary metabolite and prodrug of muscimol in A. muscaria, which also crosses the blood-brain barrier.²⁰ It is an agonist of N-methyl-D-aspartate (NMDA) receptor (NM DAR) and trans-1-amino-1,3-dicarbosycyclopentane (metabotropic quisqualate Q) receptors.⁶ In 1992, Zinkand et al. observed that ibotenic acid's neurotoxicity stems from its activation of NMDAR.²¹ The proper physiological functioning of the NMDA receptor is critical for maintaining synaptic plasticity and memory function.

The toxic effects of NMDAR activation buy ibotenic acid center around the movement of calcium ions in and out of neuronal cells. Usually, the NMDAR allows calcium ions to enter a cell upon activation by the endogenous ligand N-methyl-D-aspartate. This mechanism is tightly controlled. However, the binding of ibotenic acid to NMDAR allows excess calcium ions to flow into the cells, disrupting homeostasis and causing cell death via several mechanisms.²² For example, calcium ions activate the enzyme calmodulin kinase inside the cell, which phosphorylates (and thus activates) several enzymes. Excess calcium ions potentiate this reaction and catalyze the formation of excess reactive oxygen species that damage the cell.

In the lab, ibotenic acid is used to create brain lesions in rodents.^{23,24} It is also used in creating a rodent model for studying Alzheimer's disease.²⁵

Studies indicate that the compounds from A. muscaria shown below are biologically active in some way. There are likely more effects and more active compounds besides these. The literature refers to some compounds as ‘minor.’

In 1967, Waser self-tested muscimol and reported confusion, colored visual distortions, difficulty finding the right words, and eventually sleep.²⁶ Tamminga et al. have suggested that muscimol could be used to treat schizophrenia.²⁷ Clinical studies indicate muscimol has a variety of effects including antispastic, analgesic, anxiolytic, and as a muscle relaxant (summarized in Krogsgaard-Larsen et al., 1985). 28 Other studies suggest that muscimol may be effective in treating essential tremor,²⁹ epilepsy,³⁰ and tardive dyskinesia that patients experience in conjunction with schizophrenia.^{31, 32} In some of these studies, the effects were elicited at doses below those causing psychotropic effects. Stebelska stated, “Less poisonous muscimol and its derivatives possess high therapeutic potential in neurology and psychiatry, although the potential is still insufficiently put to good account.” ⁶

After self-administration of ibotenic acid, Waser reported sleep, lassitude, migraine headache (lasting two weeks), and one-sided visual disturbances.²⁶ Chilton reported dizziness, unsteadiness, narrowed field of vision, mild visual spasms, muscular twitches, and sleep after ingesting ibotenic acid.³³ The symptoms of ibotenic acid poisoning include nausea and drowsiness, progressing to confusion, euphoria, visual distortions, sensation of floating, sense of greater physical strength, distractibility, retrograde amnesia, and visual hallucinations including brightly lit tunnels and swirling colors.¹⁰ Administration of pure ibotenic acid to animals can cause convulsions.³⁴ Injection directly into the brain of animals causes lesions at the injection site and results in seizures.³⁵

Acetylcholine (ACh) is a neurotransmitter with a variety of effects in cells, the central and peripheral nervous systems, and neuromuscular junctions.³⁶ Atropine is a non-selective competitive antagonist with ACh at muscarinic receptors.

Amatoxins and phallotoxins (minor compounds) are slow and fast-acting toxins, respectively. The LD₅₀ of amatoxin is around 0.1 mg/kg in humans.³⁷ This amount may be present in a single A. muscaria mushroom.

Amavadin contains the heavy metal vanadium. Researchers hypothesize that amavadin may act as a peroxidase by utilizing hydrogen peroxide also present in A. muscaria.³⁸⁻⁴⁰ This mechanism may also help the mushroom self-regenerate damaged tissues.

Atropine (minor compound) is a drug used primarily in ophthalmology for pupil dilation and heart disease for increasing slow heart rate.⁴¹ Atropine is also used as an antidote for muscarine poisoning.⁴²

Betalamic acid is a potent free radical scavenger especially at pH>5.⁴³

β(1→6)-branched (1→3)-β-d-glucan showed significant antitumor activity against sarcoma 180 tumors in mice.⁴⁴ Further, mixing it with the chemotherapy drug mitomycin C caused an antitumor effect greater than that of mitomycin C alone.

In a 2013 study, fucomannogalactan significantly reduced the inflammatory pain in mice induced by formalin.⁴⁵

According to Michelot and Melendez-Howell, 2003, mushrooms, in general, are known to accumulate a variety of heavy metal compounds from the soil, some of which are toxic.¹ A. muscaria is unique in that it may concentrate vanadium as high as 200 ppm, which is 30X what is reported for any other organism. A. muscaria also has high levels of cadmium, cobalt, chromium, lead, mercury, and nickel.

Hyoscyamine (minor compound) is an antagonist of muscarinic ACh receptors. 46 It is the levorotary isomer of atropine (aka levo-atropine). Used to relieve spasms for people with peptic ulcers, irritable bowel syndrome, colic, diverticulitis, and pancreatitis. According to the summary at Drugbank.ca, it is also used to treat heart conditions and control some symptoms of Parkinson's disease.

In A. muscaria, L-DOPA is used along with L-tyrosine and the enzyme 3,4-dihydroxyphenylalanine-4,5-dioxygenase to synthesize betalamic acid.^{47, 48} L-DOPA itself is a prodrug of dopamine used to treat Parkinson's disease.⁴⁹

Muscarine (minor compound) is an agonist of muscarinic acetylcholine receptors. Fraser reported that pure crystalline muscarine chloride prepared from A. muscaria caused spasms in muscles of the gut, uterus, urinary bladder, and bronchus of several species both in vitro and in vivo. 5° It also caused a drop in blood pressure in the rabbit, cat, and dog that was 4X more potent than ACh. Also, the response to muscarine was slower and lasted longer than ACh. Muscarine was 4X more active in (temporarily) decreasing respiratory movements in the cat and rabbit. Muscarine was not hydrolyzed by cholinesterase enzymes, which may account for its prolonged activity compared to ACh. It did not cause paralysis of the neuromuscular junctions of the rat diaphragm or the cat gastrocnemius. Atropine is an antidote for muscarine poisoning. Fraser observed that muscarine “was inactive by mouth in a monkey in a quantity many times that which would cause poisoning by ingestion of Amanita muscaria in the human.”

The effects of muscazone are similar to muscimol but less potent.⁵ It is pharmacologically less active than muscimol or ibotenic acid.

Phallolysin is a compound consisting of three high molecular weight proteins and lectin.⁵¹ It has characteristics similar to the staphylococcal α-toxin. It causes cytolysis is several types of mammalian cells, including red blood cells. The protein and lectin fractions are individually cytotoxic to murine lymphocytic leukemia cell line L1210.⁵²

Scopolamine (minor compound) is a potent anticholinergic drug. A review by Safer and Allan says, “Its major central effects include an impairment in ability to retain new information, inability to fix attention, auditory and visual hallucinations, disorientation, euphoria, a feeling of fatigue, and motor incoordination. Sedation occurs following only low and moderate doses, and restlessness occurs only in higher doses.”⁵³ Scopolamine is used to induce dementia, amnesia, and short-term memory loss in laboratory animals.⁵⁴⁻⁵⁶

Atropine is an antidote for muscarine poisoning but not for A. muscaria poisoning. The progression of symptoms of A. muscaria poisoning from muscarine-like to atropine-like suggests an entourage effect. Some of the overall effects could be due to contributions from both mechanistic pathways. Duffy gives such an example.¹⁰ Muscimol competes with GABA for GABA A R binding sites. Thus, GABA cannot bind to cause a regulatory suppressive effect on neuronal activity. This results in increased neuronal excitation and synthesis of glutamate and other excitatory amino acids. At the same time, ibotenic acid may exert an excitatory effect on receptors similar to that of glutamic acid. This effect becomes even more severe with the simultaneous loss of neuronal inhibition caused by muscimol. The result is more pronounced excitatory effects including muscle spasms, seizures, convulsions, pupil dilation, etc.

From the data analysis in his 2012 thesis, Feeney observed that nausea and vomiting decreased when A. muscaria was consumed as a hot or cold tea, compared to ingesting the dried mushroom flesh or consuming the solids with the tea.⁵⁷ Statistical analysis indicated that this type of preparation was a significant factor for predicting the likelihood of nausea or vomiting. “A preparation of tea reduced the odds of experiencing nausea over a fresh preparation by 53% (p<0.05).” Also, “Drinking a tea also reduced the odds of experiencing vomiting over a fresh preparation by 86% (p<0.001).” These data suggest that the compound(s) in A. muscaria that cause nausea and vomiting are not water soluble.

A. muscaria poisoning causes dry mouth (atropine-like effect), while muscarine poisoning causes excess salivation. Headaches are not a hallmark of A. muscaria ingestion,¹⁰ however, Waser experienced a prolonged migraine headache after ingesting pure ibotenic acid. 26 Waser did not report hallucinations after taking either muscimol or ibotenic acid. 26 However, hallucinations are a symptom of A. muscaria poisoning. Chilton did not experience hallucinations after ingesting ibotenic acid.³³ In 1979, Maggi and Enna proposed that “the behavioral and biochemical effects observed after the systemic administration of muscimol may not be solely due to the presence of muscimol in [the] brain but rather may be related to the combined action of muscimol and some derivative or to the derivative alone.”⁵⁸

### SUMMARY

The disclosure relates to compositions comprising two purified Amanita muscaria compounds, a first purified Amanita muscaria compound and a second purified Amanita muscaria compound. The disclosure further relates to compositions comprising, consisting essentially of, or consisting of at least two purified Amanita muscaria compounds chosen from the following: ibotenic acid, muscimol, muscazone, stizolobic acid, stizolobinic acid, muscarine, muscarufin, muscaflavin, betalamic acid, muscapurpurin, muscaaurins, amavadin, acetylcholine, atropine, hyoscyamine, L-3,4-dihydroxyphenylalanine (L-DOPA), phallolysin, scopolamine, fucomannogalactan, β(1→6)-branched (1→3)-β-d-glucan, amatoxins, phallotoxins, or the salts of these Amanita muscaria compounds. In a composition of the disclosure the molar ratio of the first purified Amanita muscaria compound to the second Amanita muscaria compound in the composition is from 0.1:100 to 100:0.1, from 1:100 to 100:1, from 1:50 to 50:1, from 1:25 to 25:1, from 1:20 to 20:1, from 1:10 to 10:1, from 1:5 to 5:1, from 1:2 to 2:1 or is 1:1.

The disclosure also relates to formulations comprising a composition comprising, consisting essentially of, or consisting of at least two purified Amanita muscaria compounds and an excipient. The formulation may be a pharmaceutical formulation comprising a composition of the disclosure and a pharmaceutically acceptable excipient, where the first purified Amanita muscaria compound and the second Amanita muscaria compound are each present in a therapeutically effective amount. In one embodiment a pharmaceutical formulation may further comprise a therapeutically effective amount of a serotonergic drug, a purified psilocybin derivative, a purified cannabinoid, or a purified terpene.

Also disclosed are methods of regulating the activity of a neurotransmitter receptor by administering to a person in need thereof an effective dose of a composition of the disclosure or of a formulation of the disclosure or by administering to a person in need thereof a pharmaceutical formulation of the disclosure.

The disclosure also provides methods of treating a psychological disorder, a compulsive disorder, or a depressive disorder by administering to a person in need thereof an effective dose of a composition of the disclosure or of a formulation of the disclosure or by administering to a person in need thereof a pharmaceutical formulation of the disclosure.

### DETAILED DESCRIPTION

Disclosed herein are new compositions and formulations of purified Amanita muscaria compounds as well as methods for the therapeutic use of those compositions and formulations. Compositions, formulations and methods of the disclosure have at least a first Amanita muscaria compound, and a second Amanita muscaria compound, such that the first Amanita muscaria compound is different from the second Amanita muscaria compound, and the combination is substantially free from another Amanita muscaria compound. Non-naturally occurring Amanita muscaria compound compositions and formulations are disclosed. In each the Amanita muscaria compounds are combined into compositions and formulations via human ingenuity to arrive at compositions and formulations that are not found in nature. These compositions and formulations have different physical properties from those found in nature and provide different pharmacological properties. In many cases, the disclosed compositions and formulations provide different clinical effects when administered to a subject.

This disclosure provides a composition comprising, consisting essentially of, or consisting of a first purified Amanita muscaria compound and a second purified Amanita muscaria compound. Plant extracts per se do not include “purified” compounds. The first purified Amanita muscaria compound is different from the second purified Amanita muscaria compound meaning that the Amanita muscaria compound molecule of the “first purified Amanita muscaria compound” is structurally different from that of the “second purified Amanita muscaria compound.” Such structural differences, could be determined and demonstrated by a variety of analytical methods known in the chemical arts, including but not limited to chromatography, NMR, x-ray crystallography, etc. A composition of the disclosure may be prepared using techniques known in the art such as mixing of a first purified Amanita muscaria compound and a second purified Amanita muscaria compound, forming a solution or slurry of a first purified Amanita muscaria compound and a second purified Amanita muscaria compound followed by solvent removal and other such techniques.

A composition of the disclosure comprises, consists essentially of or consists of two purified Amanita muscaria compounds chosen from the following: ibotenic acid, muscimol, muscazone, stizolobic acid, stizolobinic acid, muscarine, muscarufin, muscaflavin, betalamic acid, muscapurpurin, muscaaurins (R can be ibotenic acid, stizolobic acid, aspartic acid, glutamic acid, histadine, and other aminoacids), acetylcholine, atropine, hyoscyamine, L-3,4-dihydroxyphenylalanine, phallolysin, scopolamine, fucomannogalactan, β(146)-branched (143)-β-d-glucan, amatoxins, phallotoxins, or the salts of these Amanita muscaria compounds. The structural formulas of Amanita muscaria compounds is shown below. Each is naturally present in many naturally occurring organisms, including Amanita muscaria, and occurs as a minor component within a mixture of many other molecules.

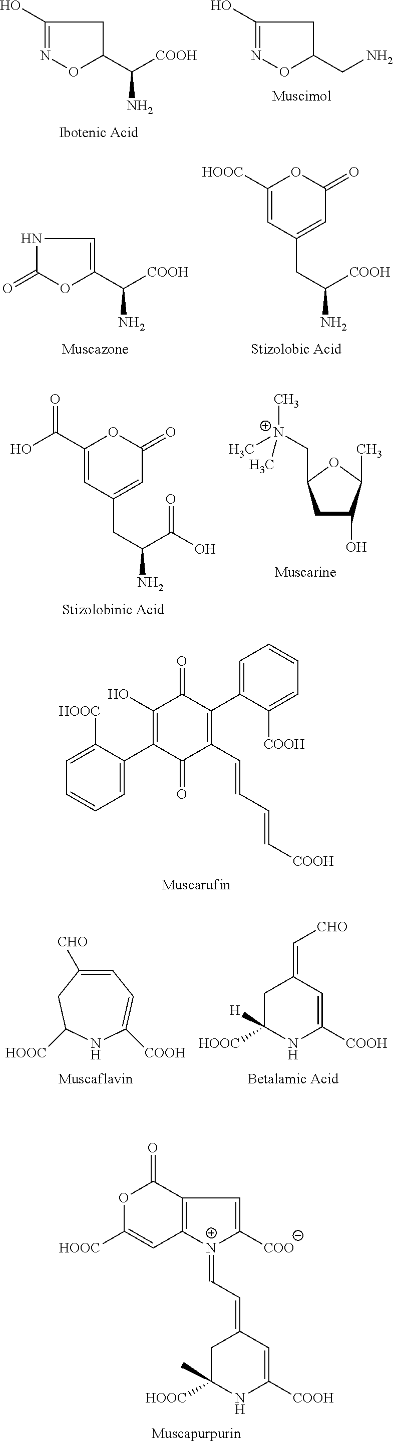

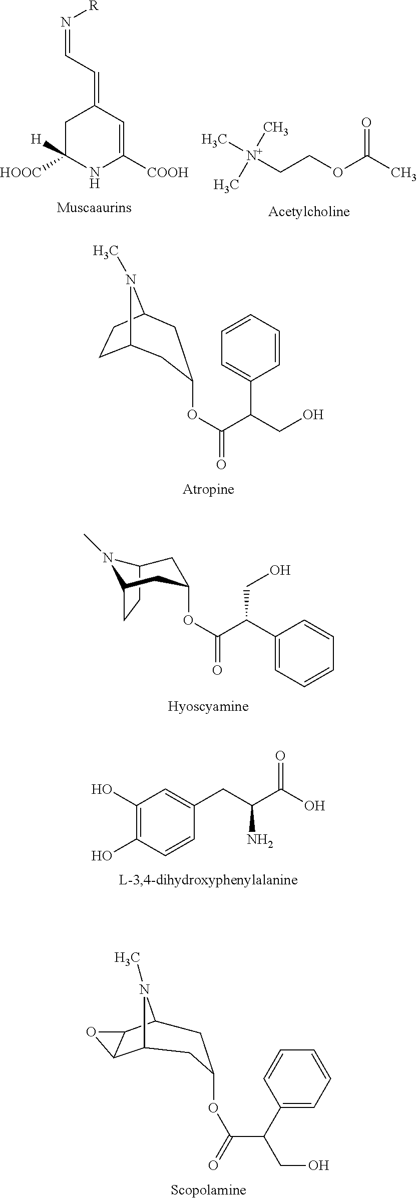

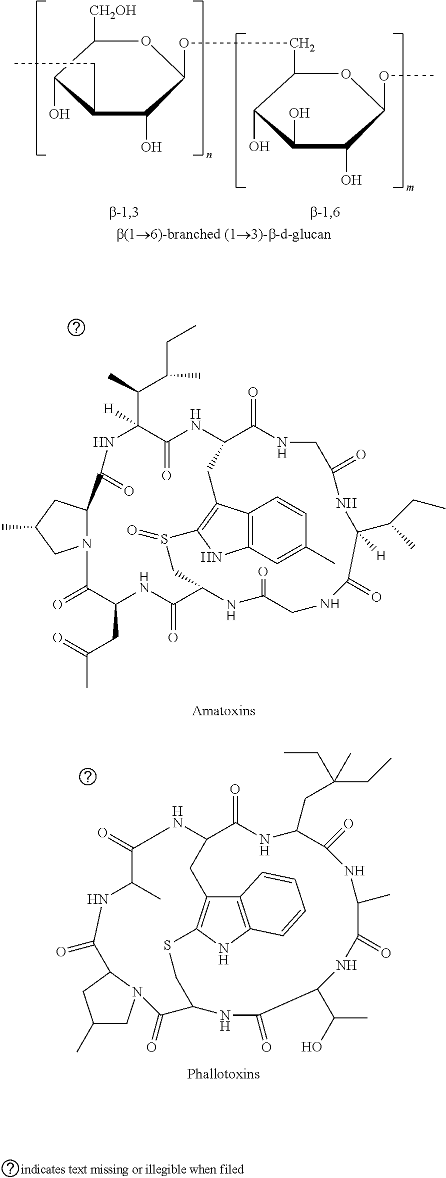

A composition of the disclosure contains a first purified Amanita muscaria compound and a second purified Amanita muscaria compound, wherein the first purified Amanita muscaria compound is different from the second purified Amanita muscaria compound. A composition of the disclosure comprises, consists essentially of or consists of two purified Amanita muscaria compounds chosen from the following: ibotenic acid, muscimol, muscazone, stizolobic acid, stizolobinic acid, muscarine, muscarufin, muscaflavin, betalamic acid, muscapurpurin, muscaaurins, acetylcholine, atropine, hyoscyamine, L-3,4-dihydroxyphenylalanine, phallolysin, scopolamine, fucomannogalactan, β(1→6)-branched (1→3)-β-d-glucan, amatoxins, phallotoxins, or the salts of these Amanita muscaria compounds. Each separate composition of two of these purified Amanita muscaria compounds or a salt thereof is a separate embodiment of the disclosure. The composition comprises, consists essentially of, or consists of purposefully chosen amounts of at least two of these purified Amanita muscaria compounds or a salt thereof. A purified Amanita muscaria compound may be prepared by extracting a desired Amanita muscaria compound from its naturally occurring source or by chemical synthesis. When extracting from a naturally occurring source, the Amanita muscaria compound may be then separated from other naturally occurring compounds and from any heavy metal or heavy metal compounds found in the naturally occurring source. A composition of the invention may be made by removing one or more Amanita muscaria compounds and other compounds from a naturally occurring source such that the resulting non-natural composition contains the desired Amanita muscaria compounds, those that remain after other Amanita muscaria compounds and other compounds are removed. For example, ibotenic acid may be removed from Amanita muscaria and then used to prepare a composition of the disclosure.

For example, purified β(1→6)-branched (1→3)-β-d-glucan and the protein components of phallolysin may be combined into a formulation for treating tumors. Muscimol may be combined with fucomannogalactan for treating pain.

Exemplary molar ratios of the first purified Amanita muscaria compound to the second Amanita muscaria compound in a composition of the disclosure include but are not limited to from about 0.1:100 to about 100:0.1, from about 1:100 to about 100:1, from about 1:50 to about 50:1, from about 1:25 to about 25:1, from about 1:20 to about 20:1, from about 1:10 to about 10:1, from about 1:5 to about 5:1, from about 1:2 to about 2:1 or may be about 1:1. Where a purposefully chosen composition contains more than two Amanita muscaria compounds each Amanita muscaria compound within the purposefully chosen composition will be present in a molar ratio such as just mentioned with respect to each other Amanita muscaria compound present. A composition containing more than two purified Amanita muscaria compounds may then be considered as a composition or mixture of first and second purified Amanita muscaria compounds. For example, a composition may include a first purified Amanita muscaria compound, a second purified Amanita muscaria compound and a third purified Amanita muscaria compound selected from the same Amanita muscaria compounds as the first and second, wherein the first purified Amanita muscaria compound is different from each of the second purified Amanita muscaria compound and the third Amanita muscaria compound. Or, a composition may include a first purified Amanita muscaria compound, a second purified Amanita muscaria compound and a third and a fourth purified Amanita muscaria compound selected from the same Amanita muscaria compounds as the first and second, wherein the first purified Amanita muscaria compound is different from each of the second purified Amanita muscaria compound and the third and fourth Amanita muscaria compound.

In one embodiment of a composition of the disclosure the first purified Amanita muscaria compound and the second purified Amanita muscaria compound is selected from: ibotenic acid, muscimol, muscazone, stizolobic acid, stizolobinic acid, muscarine, muscarufin, muscaflavin, betalamic acid, muscapurpurin, muscaaurins, acetylcholine, atropine, hyoscyamine, L-3,4-dihydroxyphenylalanine, phallolysin, scopolamine, fucomannogalactan, β(16)-branched (13)-β-d-glucan, amatoxins, and phallotoxins. Or, in other compositions of the disclosure the first purified Amanita muscaria compound and the second purified Amanita muscaria compound is selected from ibotenic acid, muscimol, muscazone, stizolobic acid, stizolobinic acid, muscarine, muscarufin, muscaflavin, betalamic acid, muscapurpurin, and muscaaurins.

In another embodiment, increasing the amount of at least a single Amanita muscaria compound in the whole fungus extract may elicit or increase the desired effect. For example, adding additional muscimol may be used for treating schizophrenia, causing muscle relaxation, or relieving pain. Adding additional fucomannogalactan may reduce inflammatory pain, such as in rheumatoid arthritis. Adding additional L-DOPA may be useful for treating Parkinson's disease. Adding additional hyoscyamine may be useful for relieving spasms in the gastrointestinal track.

In a further embodiment, removing at least a single Amanita muscaria compound from the whole fungus extract may reduce or eliminate an undesirable effect. For example, ibotenic acid may be removed to reduce convulsions, seizures, and toxicity. Amatoxins (and/or phallotoxins) may be removed to reduce toxicity. Accordingly, in certain embodiments are described compositions comprising an Amanita muscaria extract that is substantially free of ibotenic acid. In certain embodiments, the extract comprises a purified extract. In certain embodiments, the composition comprises muscimol (e.g., purified muscimol) substantially free of ibotenic acid. In other embodiments, muscimol (or muscazone or scopolamine, or all three) may be removed to reduce psychoactive effects. Muscarine may be removed to eliminate the atropine-like poisoning effects.

In certain embodiments a composition of the disclosure comprises a ratio of a first purified Amanita muscaria compound to the second Amanita muscaria compound in the composition is from 0.1:100 to 100:0.1, from 1:100 to 100:1, from 1:50 to 50:1, from 1:25 to 25:1, from 1:20 to 20:1, from 1:10 to 10:1, from 1:5 to 5:1, from 1:2 to 2:1 or is 1:1. In certain embodiments, the first purified Amanita muscaria compound comprises ibotenic acid, and the second purified Amanita muscaria compound comprises muscimol. In certain embodiments, the ratio of muscimol: ibotenic acid is greater than 5:1. In certain embodiments, the ratio of muscimol: ibotenic acid is from 5:1 to 1000:1, such as about 6:1 to about 100:1 or about 10:1 to about 50:1. In certain embodiments, the ratio of muscimol: ibotenic acid is from 15:1 to 500:1. In certain embodiments, the ratio of muscimol: ibotenic acid is from 20:1 to 400:1. In certain embodiments, the ratio of muscimol: ibotenic acid is from 25:1 to 300:1. In certain embodiments, the ratio of muscimol: ibotenic acid is from 30:1 to 250:1.

In certain embodiments, a composition comprising a first purified Amanita Muscaria compound and a second Amanita Muscaria compound is described. In some embodiments, the first compound comprises muscimol. In some embodiments, the second compound comprises ibotenic acid. In certain embodiments, the composition comprises an Amanita Muscaria extract. In certain embodiments, ibotenic acid comprises less than 10 wt. % of the composition. In certain embodiments, ibotenic acid comprises less than 8 wt. % of the composition. In certain embodiments, ibotenic acid comprises less than 5 wt. % of the composition. In certain embodiments, ibotenic acid comprises less than 3 wt. % of the composition. In certain embodiments, ibotenic acid comprises less than 1.0 wt. % of the composition. In certain embodiments, ibotenic acid comprises less than 0.5 wt. % of the composition. In certain embodiments, ibotenic acid comprises less than 0.25 wt. % of the composition.

Disclosed herein are formulations, such as pharmaceutical formulations, utilizing a purposefully chosen composition of two or more individual purified Amanita muscaria compounds and optionally an inactive compound, such as an excipient. A pharmaceutical formulation contains a therapeutically effective amount of each individual purified Amanita muscaria compound. A therapeutically effective amount of each individual purified Amanita muscaria compound is an amount which correlates to a therapeutic effect and may separately range from, for example, about 0.5-about 200 mg, about 1 mg-about 100 mg, about 2 mg-about 50 mg, about 5 mg-about 25 mg or 25 mg. The actual amount required for treatment of any particular disease, disorder or condition for any particular patient may depend upon a variety of factors including, for example, the particular disease, disorder or condition being treated; the disease state being treated and its severity; the specific pharmaceutical composition employed; the age, body weight, general health, sex and diet of the patient; the mode of administration; the time of administration; the route of administration; and the rate of excretion; the duration of the treatment; any drugs used in combination or coincidental with the specific compound employed; and other such factors well known in the medical arts. The total amount of purified Amanita muscaria compounds in a formulation may range from about 0.01 to 100 wt. %, from about 0.1 to 100 wt. %, from about 1 to about 99 wt. %, from about 50 to about 90 wt. %, from about 5 to about 75 wt. %, from about 10 to about 50 wt. %, from about 10 to about 25 wt. %, or from about 15 to about 40 wt. %.

A composition of the invention may be formulated in any type or pharmaceutical formulation known in the art. A pharmaceutical formulation of the disclosure may be solid dosage form such as an oral dosage form, e.g., a pill, capsule, and the like, which may or may not be enterically coated. A composition of the disclosure may also be formulated as a pharmaceutical formulation designed to avoid first-pass metabolism. Accordingly, to avoid first pass metabolism, a composition of the invention may be formulated as a transdermal formulation, a sublingual formulation, a buccal formulation, an intravenous (I.V.) formulation, a subcutaneous (S.C.) formulation or an inhalation formulation.

A formulation of a composition of the disclosure may contain additional inactive compounds, such as excipients, binders, stabilizers, permeation enhancers, solubilizers, etc. as known in the art. For example in a pharmaceutical formulation a composition of the disclosure may be admixed with at least one pharmaceutically acceptable excipient such as, for example, sodium citrate or dicalcium phosphate or (a) fillers or extenders, such as, for example, starches, lactose, sucrose, glucose, mannitol, and silicic acid, (b) binders, such as, for example, cellulose derivatives, starch, alginates, gelatin, polyvinylpyrrolidone, sucrose, and gum acacia, (c) humectants, such as, for example, glycerol, (d) disintegrating agents, such as, for example, agar-agar, calcium carbonate, potato or tapioca starch, alginic acid, croscarmellose sodium, complex silicates, and sodium carbonate, (e) solution retarders, such as, for example, paraffin, (f) absorption accelerators, such as, for example, quaternary ammonium compounds, (g) wetting agents, such as, for example, cetyl alcohol, and glycerol monostearate, magnesium stearate and the like (h) adsorbents, such as, for example, kaolin and bentonite, and (i) lubricants, such as, for example, talc, calcium stearate, magnesium stearate, solid polyethylene glycols, sodium lauryl sulfate, or mixtures thereof. In the case of capsules, tablets, and pills, the dosage forms may also comprise buffering agents. Pharmaceutically acceptable adjuvants known in the pharmaceutical formulation art may also be used in the pharmaceutical compositions of the invention. These include, but are not limited to, preserving, wetting, suspending, sweetening, flavoring, perfuming, emulsifying, and dispensing agents.

A pharmaceutical formulation of the disclosure may contain an additional active compound selected from the group consisting of: [2M-H] adipyl arginine, [2M-H] pimeloyl arginine, [2M-H] suberoyl arginine, 1-beta-hydroxybufalin, 1-methyl-2,9-dihydro-1H-pyrido[3—b]indo1-6-ol, 11α-hydroxy hellebrigenin, 11α-hydroxymarinobufagin, 11α-hydroxymarinobufagin, 11α-hydroxytelocinobufagin, 11a—hydroxytelo cinobufagin, Marinosin, 11a,19-dihydroxymarinobufagin, 12R-hydroxycinobufagin, 12R-hydroxytetrahydroresibufogenin 3-sulfate,15-hydroxybufalin, 16-desacetyl-19-oxocinobufotalin, 16-desacetylcinobufaginol, 19-hydroxybufalin, 19-hydroxybufalin 3-suberoyl-L-3-methylhistidine ester, 19-hydroxybufalin 3-suberoyl-L-histidine ester, 19,hydroxycinobufotalin, 19-hydroxytelocinobufagin, 19-hydroxytelocinobufagin, 19-oxobufalin, 19-oxocinobufagin, 19-oxocinobufotalin, 19-oxodesacetylcinobufagin, 20,21-epoxyresibufagin, 20R,21-epoxyresibufogenin, 205,21-epoxyresibufogenin, 1,2,3,4-tetrahydro-6-hydroxy-carbolise, 2-methyl-6-hydroxy-1,2,3,4-tetrahydro-R-carboline, 3-((N-azelayl argininyl) marinobufagin), 3-(N-adipoyl argininyl) marinobufagin, 3-(N-azelayl argininyl)-bufalin, 3-(N-dodecadienoyl argininyl) marinobufagin, 3-(N-glutaryl argininyl)-marinobufagin, 3-(N-pimeloyl argininyl) marinobufagin, 3-(N-pimeloyl argininyl) telocinobufagin, 3-(N-sebacyl argininyl) marinobufagin, 3-(N-sebacyl argininyl) telocinobufagin, 3-(N-sebacyl argininyl)-bufalin, 3-(N-suberoyl argininyl) hellebrigenin, 4-amido-3-hydroxymethyl-cyclooctylamidezotetra-alpha-furanone 5-hydroxytryptophan, 5-hydroxy-1H-indole-3-carbaldehyde, 5-hydroxyindoleacetic acid, 5-hydroxytryptophol, 5-methoxytryptophol, 5-methoxyindoleacetic acid, bufothionine, dehydrobufotenine, dehydrobufoteninehydrobromide, O-methylnordehydrobufotenine, 6-hydroxy-1-oxo-3,4-dihydro-p-carboline, Marinobufotoxin, Telocinobufatoxin, Bufalitoxin, 3-(N-undecadienoyl argininyl) marinobufagin, 3-beta-formyloxyresibufogenin, 3-O-formyl-20R,21-epoxy resibufogenin, 3-oxo-20S,21-epoxyresibufogenin, 4R-hydroxybufalin, 5Z,9Z-3-(1-hydroxybutyI)-5-propylindolizidine, 6-alpha-hydroxycinobufagin, 6Z,10E-4,6-di(pent-4-enyl) quinolizidine, adenine, arenobufagin, arenobufagin 3-sulfate, arenobufagin hemisuberate, arenobufotoxin, argentinogenin, argininesuberoyl marinobufagenin, azelayl arginine, bacagin, BLP1, BLP2, BLP3, bufalin, bufalon, bufotalone, 5-hydroxycinobufaginol, arenobufagin-3-hemisuberate, cinobufotalin-3-hemisuberate,desacetylbufotalin, bufotalin-3-suberoyl-arginineester, bufalin-3-suberoyl-arginineester, cinobufagin-3-suberoyl-arginineester, Resibufogenin-3-suberoyl-arginineester, 11-Hydroxyhellebrigenol, 5-Hydroxygambufotalin, 5,11-Hydroxyresibufaginol, Bufarenogin, 4)-Bufarenogin, 5-Hydroxyarenobufagin, Hellebrigenol-3-X1, Hellebrigenol-3-X2, Hellebrigenol-3-X3, 12-Hydroxyresibufogenin, 5-Hydroxybufotalin, 12, oxobufalin, 3-Acetylresibufogenin,19-oxoresibufagin, 1-Hydroxybufalin, Bufalin-3β-acrylic ester, Telocinobufagin-3-hemisuberate, 3-0xo-cinobufotalin, Hellebrigenin-3-hemisuberate, 1-Hydroxyarenobufagin, 16-Hydroxytelocinobufagin, Cinobufotalin-3-azelaoyl-arginineester, Cinobufagin-3-succinoyl-arginineester, Bufotalin-3-succinoyl-arginineester, Hellebrigenin-3-suberoyl-arginineester, Bufalin-3-succinoyl-arginineester Telocinobufagin-3-suberoyl-arginineester, Bufotalin-3-pimeloyl-arginineester, 19-0xocinobufagin-3-adipoyl-arginineester, Telocinobufagin-3-succinoyl-arginineester, Bufalin-3-pim-1, Bufarenogin-3-suberoyl-arginineester, Desacetylcinobufaginol-3-suberoyl-arginineester, Arenobufagin-3-suberoyl-arginineester, Cinobufagin-3-hemisuberate, 12-hydroxybufalin, 16-Hydroxyhellebrigenin, Argentinogenin-3-hemisuberate, Argentinogenin-iso, 3-0xoargentinogenin, 3-Oxoarenobufagin, Hellebrigenol-3-sulfate, Hellebrigenin-3-sulfate, 19-Hydroxybufalin-3-sulfate, Telocinobufagin-3-sulfate, Arenobufagin-16-acetyl, Resibufogenin-3-formly, 513,12P-Dihydroxycinobufagin, 16)3-Acetoxybufarenogin, 11a,12P-Dihydroxybufalin, 20S,21-Epoxymarinobufagin, 20S,21-Epoxymarinobufagin-3-Acetyl, Arenobufagin-3-sulfate, Bufalin-3-adipoyl-arginineester, Bufalin-3-pimeloyl-arginineester, Bufotalin-3-sulfate, Cinobufagin-3-adipoyl-arginineester, Cinobufagin-3-glut-2, Cinobufagin-3-pimeloyl-arginineester, Cinobufagin-3-sulfate, Cinobufotalin-3-suberoyl-arginineester, Desacetylcinobufagin-3-succinoyl-arginineester, Gambufotalin-3-adipoyl-arginineester, Gambufotalin-3-pimeloyl-arginineester, Gambufotalin-3-suberoyl-arginineester, Gambufotalin-3-succinoyl-arginineester, Gambufotalin-3-sulfate, Marinobufagin-3-glut-2, Marinobufagin-3-pimeloyl-arginineester, Marinobufagin-3-glut-2, Marinobufagin-3-suberoyl-arginineester, Marinobufagin-3-succinoyl-arginineester, Resibufogenin-3-succinoyl-arginineester, Telocinobufagin-3-glut-2, 12-hydroxycinobufagin, resibufogenin-3-hemisuberate, bufalin-3-hemisuberate, cinobufagin-3-suc-1, Desacetylcinobufagin-3-suc-1, gambufotalin-3-hemisuberate, bufalin 3-adipoyl-L-arginine ester, bufalin 3-pimeloyl-L-arginine ester, bufalin 3-suberoyl-L-histidine ester, bufalin 3-succinoyl-L-arginine ester, bufalin hemisuberate, bufalin-3-sulfate, bufogargarizin A, bufogargarizin B, bufogargarizin C, bufogargarizin D, bufogenin, bufotalin, bufotalin 3-suberoyl-L-1-methylhistidine ester, bufotalin 3-suberoyl-L-3-methylhistidine ester, Vulgarobufotoxin, bufotalin 3-succinoylarginine ester(I), bufotalin 3-sulfate, bufotalinin, bufotalone, bufotoxin, caffeine, cholesterol, cinabufotalitoxin, cinabufotoxin, cinobufagin, cinobufagin 3-adipoyl-L-arginine ester, cinobufagin 3-glutaryl-L-arginine ester, cinobufagin 3-pimeloyl-L-arginine ester, Cinobufotoxin, cinobufagin 3-succinoyl-L-arginine ester, cinobufagin 3-sulfate, cinobufagin hemisuberate, cinobufaginol, cinobufotalin cinobufotalin 3-suberoyl-L-arginine ester, cyclo(pro-gly)dipeptide, desacetylcinobufagin, desacetylcinobufagin 3-hemisuccinate, desacetylcinobufagin 3-succinyl-L-arginine, desacetylcinobufotalin, Daigredorigenin, dopamine, epinephrine, Eritadenine, gamabufotalin, gamabufotalin 3-adipoyl-L-arginine ester, gamabufotalin 3-pimeloyl-L-arginine ester, Gamabufotalitoxin, gamabufotalin 3-succinoyl-L-arginine ester, gamabufotalin 3-sulfate, gamabufotalin hemisuberoate, gamabufotaliniol, gamma sitosterol, guanine, hellebrigenin, Hellebritoxin, hellebrigenol, hypoxanthine, indoleacetic acid, leucine, marinobufagin, marinobufagin 3-glytaryl-L-arginine ester, marinobufagin 3-pimeloyl-L-arginine ester, marinobufagin 3-suberoyl-L-arginine ester, marinobufagin 3-suberoyl-L-glutamine ester, marinobufagin 3-succinoyl-L-arginine ester, marinobufagin 3-sulfate, marinoic acid, morphine, N-(2-(5-hydroxy-1H-indol-3-yl)ethyl)-N-methylformamide, nicotinamide, nicotinic acid, norepinephrine, palmitic acid cholesterol ester, precoccinelline, resibufagin, resibufagin 3-sulfate, resibufaginol, resibufogenin, resibufagenin, resibufogenin 3-suberoyl-L-arginine ester, resibufogenin 3-succinoyl-L-arginine ester, resibufogenin hemisuberate, resibufotoxin, sebacyl arginine, shepherdine, suberic acid, succinic acid, telocinobufagin, Telocinobufogenin, telocinobufagin 3-glutaryl-L-arginine ester, telocinobufagin 3-suberoyl-L-arginine ester, telocinobufagin 3-suberoyl-L-glutamine ester, thiamethoxam, thymine, tricaine methanesulfonate, uracil, valine, Bombinakinin M, Maximin 1, Maximin, 2, Maximin 3, Maximin 4, Maximin 5, Maximin 6, Maximin 7, Maximin 8, Maximin 9, Maximin 10, Maximin H1, Maximin H2 Maximin H3, Maximin H4, Maximin H5, Maximin H6, Maximin H7, Maximin H8, Maximin H9, Maximin H10, Maximin H11, Maximin H12, Maximin H13, Maximin H14, Maximin H15, Maximin H16, Bradykinin, (Thr(6))-bradykinin, Bombinakinin-GAP, sleep-inducing factor (SIF), xanthine and mixtures thereof.

A pharmaceutical formulation of the disclosure may comprise a composition of the disclosure and a serotonergic drug, a purified psilocybin derivative, a purified cannabinoid, or a purified terpene, each present in a therapeutically effective amount using a purposefully engineered and unnaturally occurring molar ratios. Published US applications US 2018/0221396 A1 and US 2019/0142851 A1 disclose compositions comprising a combination of a first purified psilocybin derivative with a second purified psilocybin derivative, with one or two purified cannabinoids or with a purified terpene. The disclosures of US 2018/0221396 A1 and US 2019/0142851 A1 are incorporated herein by reference. According to this disclosure, a composition containing two purified Amanita muscaria compounds as discussed above may be used in place of a “first purified psilocybin derivative” in the compositions described in US 2018/0221396 A1 and US 2019/0142851 A1. Accordingly, the disclosure provides a pharmaceutical formulation comprising as a first component: a composition containing two purified Amanita muscaria compounds according to this disclosure and as a second component selected from (a) a purified psilocybin derivative, (b) one or two purified cannabinoids and (c) a purified terpene; with the rest being at least one suitable pharmaceutical excipient or at least one other adjuvant, as discussed below. Such a composition may be a pharmaceutical composition wherein the components are present individually in therapeutic effective amounts or by combination in a therapeutically effective amount to treat a disease, disorder, or condition as described herein.

A serotonergic drug refers to a compound that binds to, blocks, or otherwise influences (e.g., via an allosteric reaction) activity at a serotonin receptor as described in paragraphs [0245]-[0253] of US 2018/0221396 A1 and [0305]-[0311] US 2019/0142851 A1 as well as the disclosed preferred embodiments, incorporated here by reference. Some exemplary serotonergic drugs include the following molecules: 6-Allyl-N,N-diethyl-NL, N,N-Dibutyl-T, N,N-Diethyl-T, N,N-Diisopropyl-T, 5-Methyoxy-alpha-methyl-T, N,N-Dimethyl-T, 2,alpha-Dimethyl-T, alpha,N-Dimethyl-T, N,N-Dipropyl-T, N-Ethyl-N-isopropyl-T, alpha-Ethyl-T, 6,N,N-Triethyl-NL, 3,4-Dihydro-7-methoxy-1-methyl-C, 7-Methyoxy-l-methyl-C, N,N-Dibutyl-4-hydroxy-T, N,N-Diethyl-4-hydroxy-T, N,N-Diisopropyl-4-hydroxy-T, N,N-Dimethyl-4-hydroxy-T, N,N-Dimethyl-5-hydroxy-T, N, N-Dipropyl-4-hydroxy-T, N-Ethyl-4-hydroxy-N-methyl-T, 4-Hydroxy-N-isopropyl-N-methyl-T, 4-Hydroxy-N-methyl-N-propyl-T, 4-Hydroxy-N,N-tetramethylene-T Ibogaine, N,N-Diethyl-L, N-Butyl-N-methyl-T, N,N-Diisopropyl-4,5-methylenedioxy-T, N,N-Diisopropyl-5,6-methylenedioxy-T, N,N-Dimethyl-4,5-methylenedioxy-T, N,N-Dimethyl-5,6-methylenedioxy-T, N-Isopropyl-N-methyl-5,6-methylenedioxy-T, N,N-Diethyl-2-methyl-T, 2,N,N-Trimethyl-T, N-Acetyl-5-methoxy-T, N,N-Diethyl-5-methoxy-T, N,N-Diisopropyl-5-methoxy-T, 5-Methoxy-N,N-dimethyl-T, N-Isopropyl-4-methoxy-N-methyl-T, N-Isopropyl-5-methoxy-N-methyl-T, 5,6-Dimethoxy-N-isopropyl-N-methyl-T, 5-Methoxy-N-methyl-T, 5-Methoxy-N,N-tetramethylene-T, 6-Methoxy-1-methyl-1,2,3,4-tetrahydro-C, 5-Methoxy-2,N,N-trimethyl-T, N,N-Dimethyl-5-methylthio-T, N-Isopropyl-N-methyl-T, alpha-Methyl-T, N-Ethyl-T, N-Methyl-T, 6-Propyl-N L, N,N-Tetramethylene-T, Tryptamine, and 7-Methoxy-1-methyl-1,2,3,4-tetrahydro-C, alpha,N-Dimethyl-5-methoxy-T. For additional information regarding these compounds See Shulgin, A. T., & Shulgin, A. (2016). Tihkal: The Continuation. Berkeley, Calif.: Transform Press. In one embodiment, a serotonergic drug is chosen from alprazolam, amphetamine, aripiprazole, azapirone, a barbiturate, bromazepam, bupropion, buspirone, a cannabinoid, chlordiazepoxide, citalopram, clonazepam, clorazepate, dextromethorphan, diazepam, duloxetine, escitalopram, fluoxetine, flurazepam, fluvoxamine, lorazepam, lysergic acid diethylamide, lysergamide, 3,4-methylenedioxymethamphetamine, milnacipran, mirtazapine, naratriptan, paroxetine, pethidine, phenethylamine, psicaine, oxazepam, reboxetine, serenic, serotonin, sertraline, temazepam, tramadol, triazolam, a tryptamine, venlafaxine, vortioxetine, and/or derivatives thereof. In an exemplary embodiment, the serotonergic drug is 3,4-methylenedioxymethamphetamine.

Exemplary psilocybin derivatives include but are not limited to psilocybin itself and the psilocybin derivates described in paragraphs [0081]-[0109] of US 2018/0221396 A1 and [082]-[0110] US 2019/0142851 A1 as well as the disclosed preferred embodiments, incorporated here by reference. In one embodiment, the compositions disclosed herein comprise one or more purified psilocybin derivatives chosen from: [3-(2-Dimethylaminoethyl)-1H-indol-4-yl] dihydrogen phosphate, 4-hydroxytryptamine, 4-hydroxy-N,N-dimethyltryptamine, [3-(2-methylaminoethyl)-1H-indol-4-yl] dihydrogen phosphate, 4-hydroxy-N-methyltryptamine, [3-(aminoethyl)-1H-indol-4-yl] dihydrogen phosphate, [3-(2-trimethylaminoethyl)-1H-indol-4-yl] dihydrogen phosphate, and 4-hydroxy-N,N,N-trimethyltryptamine.

Exemplary cannabinoids include but are not limited to the cannabinoids described in paragraphs [0111]-[0159] of US 2018/0221396 A1 and [0112]-[0160] US 2019/0142851 A1 as well as the disclosed preferred embodiments, incorporated here by reference. Examples of cannabinoids within the context of this disclosure include the following molecules: Cannabichromene (CBC), Cannabichromenic acid (CBCA), Cannabichromevarin (CBCV), Cannabichromevarinic acid (CBCVA), Cannabicyclol (CBL), Cannabicyclolic acid (CBLA), Cannabicyclovarin (CBLV), Cannabidiol (CBD), Cannabidiol monomethylether (CBDM), Cannabidiolic acid (CBDA), Cannabidiorcol (CBD-C1), Cannabidivarin (CBDV), Cannabidivarinic acid (CBDVA), Cannabielsoic acid B (CBEA-B), Cannabielsoin (CBE), Cannabielsoin acid A (CBEA-A), Cannabigerol (CBG), Cannabigerol monomethylether (CBGM), Cannabigerolic acid (CBGA), Cannabigerolic acid monomethylether (CBGAM), Cannabigerovarin (CBGV), Cannabigerovarinic acid (CBGVA), Cannabinodiol (CBND), Cannabinodivarin (CBDV), Cannabinol (CBN), Cannabinol methylether (CBNM), Cannabinol-C2 (CBN-C2), Cannabinol-C4 (CBN-C4), Cannabinolic acid (CBNA), Cannabiorcool (CBN-C1), Cannabivarin (CBV), Cannabitriol (CBT), Cannabitriolvarin (CBTV), 10-Ethoxy-9-hydroxy-delta-6a-tetrahydrocannabinol, Cannbicitran (CBT), Cannabiripsol (CBR), 8,9-Dihydroxy-delta-6a-tetrahydrocannabinol, Delta-8-tetrahydrocannabinol (6,8-THC), Delta-8-tetrahydrocannabinolic acid (A8-THCA), Delta-9-tetrahydrocannabinol (THC), Delta-9-tetrahydrocannabinol-C4 (THC-C4), Delta-9-tetrahydrocannabinolic acid A (THCA-A), Delta-9-tetrahydrocannabinolic acid B (THCA-B), Delta-9-tetrahydrocannabinolic acid-C4 (THCA-C4), Delta-9-tetrahydrocannabiorcol (THC-C1), Delta-9-tetrahydrocannabiorcolic acid (THCA-C1), Delta-9-tetrahydrocannabivarin (THCV), Delta-9-tetrahydrocannabivarinic acid (THCVA), 10-Oxo-delta-6a-tetrahydrocannabinol (OTHC), Cannabichromanon (CBCF), Cannabifuran (CBF), Cannabiglendol, Delta-9-cis-tetrahydrocannabinol (cis-THC), Tryhydroxy-delta-9-tetrahydrocannabinol (tri0H-THC), Dehydrocannabifuran (DCBF), and 3,4,5,6-Tetrahydro-7-hydroxy-alpha-alpha-2-trimethyl-9-n-propyl-2,6-metha-no-2H-1-benzoxocin-5-methanol. In one embodiment, the purified cannabinoid is chosen from THC, THCA, THCV, THCVA, CBC, CBCA, CBCV, CBCVA, CBD, CBDA, CBDV, CBDVA, CBG, CBGA, CBGV, or CBGVA.

Exemplary terpenes include but are not limited to the terpenes described in paragraphs [0160]-[0238] of US 2018/0221396 A1 and [0161]-[0300] US 2019/0142851 A1 as well as the disclosed preferred embodiments, incorporated here by reference. In one embodiment, a purified terpene is chosen from acetanisole, acetyl cedrene, anethole, anisole, benzaldehyde, bornyl acetate, borneol, cadinene, cafestol, caffeic acid, camphene, camphor, capsaicin, carene, carotene, carvacrol, carvone, alpha-caryophyllene, beta-caryophyllene, caryophyllene oxide, cedrene, cedrene epoxide, cecanal, cedrol, cembrene, cinnamaldehyde, cinnamic acid, citronellal, citronellol, cymene, eicosane, elemene, estragole, ethyl acetate, ethyl cinnamate, ethyl maltol, eucalyptol/1,8-cineole, eudesmol, eugenol, euphol, farnesene, farnesol, fenchone, geraniol, geranyl acetate, guaia-1(10),11-diene, guaiacol, guaiol, guaiene, gurjunene, herniarin, hexanaldehyde, hexanoic acid, humulene, ionone, ipsdienol, isoamyl acetate, isoamyl alcohol, isoamyl formate, isoborneol, isomyrcenol, isoprene, isopulegol, isovaleric acid, lavandulol, limonene, gamma-linolenic acid, linalool, longifolene, lycopene, menthol, methyl butyrate, 3-mercapto-2-methylpentanal, beta-mercaptoethanol, mercaptoacetic acid, methyl salicylate, methylbutenol, methyl-2-methylvalerate, methyl thiobutyrate, beta-myrcene, gamma-muurolene, nepetalactone, nerol, nerolidol, neryl acetate, nonanaldehyde, nonanoic acid, ocimene, octanal, octanoic acid, pentyl butyrate, phellandrene, phenylacetaldehyde, phenylacetic acid, phenylethanethiol, phytol, pinene, propanethiol, pristimerin, pulegone, retinol, rutin, sabinene, squalene, taxadiene, terpineol, terpine-4-ol, terpinolene, thujone, thymol, umbelliferone, undecanal, verdoxan, or vanillin. In one embodiment, a purified terpene is chosen from bornyl acetate, alpha-bisabolol, borneol, camphene, camphor, carene, beta-caryophyllene, cedrene, cymene, elemene, eucalyptol, eudesmol, farnesene, fenchol, geraniol, guaiacol, humulene, isoborneol, limonene, linalool, menthol, beta-myrcene, nerolidol, ocimene, phellandrene, phytol, pinene, pulegone, sabinene, terpineol, terpinolene, or valencene.

Exemplary compositions of two purified Amanita muscaria compounds of the disclosure and a second compound selected from a serotonergic drug, a purified psilocybin derivative, a purified cannabinoid, or a purified terpene in exemplary molar ratios are shown in Table 1.

TABLE 1

| | Molar ratio of two | Molar ratio of two | Molar ratio of two |
|---|---|---|---|
| | purified Amanita | purified Amanita | purified Amanita |
| | muscaria | muscaria | muscaria |
| | compounds:second | compounds:second | compounds:second |
| Second Compound | compound | compound | compound |
| 3,4- | About 1:100 to | About 1:25 to | About 1:5 to |
| methylenedioxymethamphetamine | about 100:1 | about 25:1 | about 5:1 |
| Citalopram | About 1:100 to | About 1:25 to | About 1:5 to |
| | about 100:1 | about 25:1 | about 5:1 |
| Escitalopram | About 1:100 to | About 1:25 to | About 1:5 to |
| | about 100:1 | about 25:1 | about 5:1 |
| Fluoxetine | About 1:100 to | About 1:25 to | About 1:5 to |
| | about 100:1 | about 25:1 | about 5:1 |
| Paroxetine | About 1:100 to | About 1:25 to | About 1:5 to |
| | about 100:1 | about 25:1 | about 5:1 |
| Sertraline | About 1:100 to | About 1:25 to | About 1:5 to |
| | about 100:1 | about 25:1 | about 5:1 |
| [3-(2-Dimethylaminoethyl)-1H- | About 1:100 to | About 1:25 to | About 1:5 to |
| indol-4-yl] dihydrogen phosphate | about 100:1 | about 25:1 | about 5:1 |
| 4-hydroxytryptamine | About 1:100 to | About 1:25 to | About 1:5 to |
| | about 100:1 | about 25:1 | about 5:1 |
| 4-hydroxy-N,N-dimethyltryptamine | About 1:100 to | About 1:25 to | About 1:5 to |
| | about 100:1 | about 25:1 | about 5:1 |
| [3-(2-methylaminoethyl)-1H-indol- | About 1:100 to | About 1:25 to | About 1:5 to |
| 4-yl] dihydrogen phosphate | about 100:1 | about 25:1 | about 5:1 |
| 4-hydroxy-N-methyltryptamine | About 1:100 to | About 1:25 to | About 1:5 to |
| | about 100:1 | about 25:1 | about 5:1 |
| [3-(aminoethyl)-1H-indol-4-yl] | About 1:100 to | About 1:25 to | About 1:5 to |
| dihydrogen phosphate | about 100:1 | about 25:1 | about 5:1 |
| [3-(2-trimethylaminoethyl)-1H- | About 1:100 to | About 1:25 to | About 1:5 to |
| indol-4-yl] dihydrogen phosphate | about 100:1 | about 25:1 | about 5:1 |
| 4-hydroxy-N,N,N- | About 1:100 to | About 1:25 to | About 1:5 to |
| trimethyltryptamine | about 100:1 | about 25:1 | about 5:1 |
| THC | About 1:100 to | About 1:25 to | About 1:5 to |
| | about 100:1 | about 25:1 | about 5:1 |
| CBC | About 1:100 to | About 1:25 to | About 1:5 to |
| | about 100:1 | about 25:1 | about 5:1 |
| CBD | About 1:100 to | About 1:25 to | About 1:5 to |
| | about 100:1 | about 25:1 | about 5:1 |
| CBG | About 1:100 to | About 1:25 to | About 1:5 to |
| | about 100:1 | about 25:1 | about 5:1 |
| Myrcene | About 1:100 to | About 1:25 to | About 1:5 to |
| | about 100:1 | about 25:1 | about 5:1 |
| Pinene | About 1:100 to | About 1:25 to | About 1:5 to |
| | about 100:1 | about 25:1 | about 5:1 |
| Caryophyllene | About 1:100 to | About 1:25 to | About 1:5 to |
| | about 100:1 | about 25:1 | about 5:1 |
| Limonene | About 1:100 to | About 1:25 to | About 1:5 to |
| | about 100:1 | about 25:1 | about 5:1 |
| Humulene | About 1:100 to | About 1:25 to | About 1:5 to |
| | about 100:1 | about 25:1 | about 5:1 |
| Linalool | About 1:100 to | About 1:25 to | About 1:5 to |
| | about 100:1 | about 25:1 | about 5:1 |

Exemplary pharmaceutical compositions of two purified Amanita muscaria compounds of the disclosure and a second compound selected from a serotonergic drug, a purified psilocybin derivative, a purified cannabinoid, or a purified terpene and an excipient with exemplary molar ratios of two purified Amanita muscaria compounds to the second compound are shown in Table 2.

TABLE 2

| | Molar ratio of two | Molar ratio of two | Molar ratio of two |
|---|---|---|---|
| | purified Amanita | purified Amanita | purified Amanita |
| | muscaria | muscaria | muscaria |
| | compounds:second | compounds:second | compounds:second |
| Second Compound | compound | compound | compound |
| 3,4- | About 1:100 to | About 1:25 to | About 1:5 to |
| methylenedioxymethamphetamine | about 100:1 | about 25:1 | about 5:1 |
| Citalopram | About 1:100 to | About 1:25 to | About 1:5 to |
| | about 100:1 | about 25:1 | about 5:1 |
| Escitalopram | About 1:100 to | About 1:25 to | About 1:5 to |
| | about 100:1 | about 25:1 | about 5:1 |
| Fluoxetine | About 1:100 to | About 1:25 to | About 1:5 to |
| | about 100:1 | about 25:1 | about 5:1 |
| Paroxetine | About 1:100 to | About 1:25 to | About 1:5 to |
| | about 100:1 | about 25:1 | about 5:1 |
| Sertraline | About 1:100 to | About 1:25 to | About 1:5 to |
| | about 100:1 | about 25:1 | about 5:1 |
| [3-(2-Dimethylaminoethyl)-1H- | About 1:100 to | About 1:25 to | About 1:5 to |
| indol-4-yl] dihydrogen phosphate | about 100:1 | about 25:1 | about 5:1 |
| 4-hydroxytryptamine | About 1:100 to | About 1:25 to | About 1:5 to |
| | about 100:1 | about 25:1 | about 5:1 |
| 4-hydroxy-N,N-dimethyltryptamine | About 1:100 to | About 1:25 to | About 1:5 to |
| | about 100:1 | about 25:1 | about 5:1 |
| [3-(2-methylaminoethyl)-1H-indol- | About 1:100 to | About 1:25 to | About 1:5 to |
| 4-yl] dihydrogen phosphate | about 100:1 | about 25:1 | about 5:1 |
| 4-hydroxy-N-methyltryptamine | About 1:100 to | About 1:25 to | About 1:5 to |
| | about 100:1 | about 25:1 | about 5:1 |
| [3-(aminoethyl)-1H-indol-4-yl] | About 1:100 to | About 1:25 to | About 1:5 to |
| dihydrogen phosphate | about 100:1 | about 25:1 | about 5:1 |
| [3-(2-trimethylaminoethyl)-1H- | About 1:100 to | About 1:25 to | About 1:5 to |
| indol-4-yl] dihydrogen phosphate | about 100:1 | about 25:1 | about 5:1 |
| 4-hydroxy-N,N,N- | About 1:100 to | About 1:25 to | About 1:5 to |
| trimethyltryptamine | about 100:1 | about 25:1 | about 5:1 |
| THC | About 1:100 to | About 1:25 to | About 1:5 to |
| | about 100:1 | about 25:1 | about 5:1 |
| CBC | About 1:100 to | About 1:25 to | About 1:5 to |
| | about 100:1 | about 25:1 | about 5:1 |
| CBD | About 1:100 to | About 1:25 to | About 1:5 to |
| | about 100:1 | about 25:1 | about 5:1 |
| CBG | About 1:100 to | About 1:25 to | About 1:5 to |
| | about 100:1 | about 25:1 | about 5:1 |
| Myrcene | About 1:100 to | About 1:25 to | About 1:5 to |
| | about 100:1 | about 25:1 | about 5:1 |
| Pinene | About 1:100 to | About 1:25 to | About 1:5 to |
| | about 100:1 | about 25:1 | about 5:1 |
| Caryophyllene | About 1:100 to | About 1:25 to | About 1:5 to |
| | about 100:1 | about 25:1 | about 5:1 |
| Limonene | About 1:100 to | About 1:25 to | About 1:5 to |
| | about 100:1 | about 25:1 | about 5:1 |
| Humulene | About 1:100 to | About 1:25 to | About 1:5 to |
| | about 100:1 | about 25:1 | about 5:1 |
| Linalool | About 1:100 to | About 1:25 to | About 1:5 to |
| | about 100:1 | about 25:1 | about 5:1 |

The compositions and formulations disclosed herein are products of human ingenuity, i.e., made by humans and substantially different from how they are found in nature. The disclosed compositions and formulations can be distinguished from naturally occurring forms by comparing the cellular pharmacology of the disclosed formulations with that of naturally occurring forms. The disclosed compositions and formulations can be distinguished from naturally occurring forms by comparing the molar ratios of compounds within the disclosed formulations with those found in nature. The disclosed compositions and formulations can also be distinguished from naturally occurring forms by comparing the molar ratios of compounds within the disclosed formulations with reference compounds that are present alongside the said compounds when those compounds are found in nature.

At the time of this disclosure, Amanita muscaria compounds were only available within complex mixtures comprising other compounds and matter from their natural sources, i.e., plant matter and other compounds. All data indicate that the presence and amounts of psychoactive compounds within naturally occurring samples are considered highly variable. Plant extracts often do not provide the same physical, cellular, and/or clinical properties as formulations made by combining particular compounds of known purity. In contrast, each of the compositions disclosed herein differs from previously known compositions in significant ways. For example, in the disclosed compositions and formulations, the ratio of Amanita muscaria compounds (e.g., the first purified Amanita muscaria compounds to the second purified Amanita muscaria compound) or the ratio of an Amanita muscaria compound to a naturally occurring reference compound (e.g., cellulose, ligin, chlorophyll, etc.) differs from those previously disclosed or otherwise naturally occurring. This disclosure provides compositions and formulations made with known amounts of known compounds, including known amounts of Amanita muscaria compounds. Such formulations allow for administering consistent amounts of Amanita muscaria compounds, which provides the user or subject with consistent and reliable effects.

A “purified” compound is a compound that is in a pure chemical form, not as it existed in nature. A “purified” compound is a higher purity (% purity) than is found in nature. The compound may be extracted and purified by means known in the art. For example, an Amanita muscaria compound may have been chromatographed, for example by gas chromatography, liquid chromatography (e.g., LC, HPLC, etc.), flush column chromatography, etc. or subject to crystallization, distillation, or sublimation. A compound may be purified by two or more purifications steps using those techniques or a composition of those techniques. A purified compound is a compound that is 80-100% pure, 90-100% pure or 95-100% pure. Within the context of this disclosure, the term “purified” means separated from other materials, such as plant or fungal material, e.g., protein, chitin, cellulose, or water. A purified compound is substantially free of other materials. For example, a purified compound is substantially free from a second Amanita muscaria compound; substantially free from histidine; substantially free from a biological material, such as mold, fungus, plant mater, or bacteria; or substantially free from a different unwanted compound, e.g., a compound correlated with unwanted side effects.

The disclosure also provides methods of regulating the activity of a neurotransmitter receptor by administering to a person in need thereof an effective dose of a composition of the disclosure or administering to a person in need thereof a formulation of the disclosure. The methods disclosed herein comprise treating a psychological disorder, e.g., an anxiety disorder, a compulsive disorder (e.g., an addiction), a depressive disorder, etc., with a disclosed composition or a disclosed pharmaceutical formulation. In one embodiment, the methods disclosed herein comprise treating a psychological disorder, e.g., an anxiety disorder, a compulsive disorder (e.g., and addiction), a depressive disorder, etc., by administering to a subject in need of treatment one or more of the compositions disclosed herein and a neurotransmitter activity modulator, e.g., a serotonergic drug, a dopaminergic drug, etc. The psychological disorder may be chosen from depression, psychotic disorder, schizophrenia, schizophreniform disorder (acute schizophrenic episode); schizoaffective disorder; bipolar I disorder (mania, manic disorder, manic-depressive psychosis); bipolar II disorder; major depressive disorder; major depressive disorder with psychotic feature (psychotic depression); delusional disorders (paranoia); Shared Psychotic Disorder (Shared paranoia disorder); Brief Psychotic disorder (Other and Unspecified Reactive Psychosis); Psychotic disorder not otherwise specified (Unspecified Psychosis); paranoid personality disorder; schizoid personality disorder; schizotypal personality disorder; anxiety disorder; social anxiety disorder; substance-induced anxiety disorder; selective mutism; panic disorder; panic attacks; agoraphobia; attention deficit syndrome, post-traumatic stress disorder (PTSD), premenstrual dysphoric disorder (PMDD), and premenstrual syndrome (PMS).

### REFERENCES

1. Michelot D, Melendez-Howell L M. Amanita muscaria: chemistry, biology, toxicology, and ethnomycology. Mycological Research. 2003; 107(2):131-146. doi:10.1017/S0953756203007305.
2. Borthwick P W, Steward E G. Ibotenic acid: further observations on its conformational modes. Journal of Molecular Structure. 1976;33(4141-144. doi:10.1016/0022-2860(76)80155-X.
3. Johnston G, Curtis D, DeGroat W, Duggan A. Central Actions of Ibotenic Acid and Muscimol. Biochemical Pharmacology. 1968; 17(12):2488-2489. doi:10.1016/0006-2952(68)90141-x.
4. Bowden K, Drysdale A. A novel constituent of Amanita muscaria. Tetrahedron Letters. 1965; 12:727-728. doi:10.1016/s0040-4039(01)83973-3.
5. Catalfomo P, Eugster C. Amanita muscaria—Present understanding of its chemistry. United Nations: Office on Drugs and Crime UNODC—Bulletin on Narcotics. 1970; (1ssue 4-004). Accessed Jun. 1, 2020. https://www.unodc.org/unodc/en/data-and-analysis/bulletin/bulletin_1970-01-01_4_page005.html.
6. Stebelska K. Fungal hallucinogens psilocin, ibotenic acid, and muscimol: analytical methods and biologic activities. Therapeutic Drug Monitoring. 2013; 35(4):420-442. doi:10.1097/FTD.0b013e31828741a5.
7. Theobald W, Buch 0, Kinz H, Krupp P, Stenger E, Heimann H. Pharmacological and experimental investigations of two components of the fly agaric. Arzneim Forsch. 1968; 18:311-315.
8. Merova B, Ondra P, Stankova M, \Mika I. Isolation and identification of the Amanita muscaria and Amanita pantherina toxins in human urine. Neuroendocrinology Letters. 2008; 29(5):744-748. http://www.nel.edu/userfiles/articlesnew/NEL290508A27.pdf.
9. Magdalan J, Antoriczyk A. Zatrucie muchomorem plamistym czy udar mozgu (Toadstool poisioning or stroke). Przeglqd Lekarski. 2007; 64:4-5.
10. Duffy T. Toxic Fungi of Western North America. MykoWeb; 2008. Accessed Jul. 1, 2020. https://fungi.fr/pdf/toxic_fungi.pdf.
11. Ebert B, Thompson S A, Saounatsou K, Mckernan R, Krogsgaard-Larsen P, Wafford K A. Differences in agonist/antagonist binding affinity and receptor transduction using recombinant human gammaaminobutyric acid Type A receptors. Molecular Pharmacology. 1997; 52(6):1150-1156. doi:10.1124/mo1.52.6.1150.
12. Beaumont K, Chilton W S, Yamamura H I, Enna S J. Muscimol binding in rat brain: Association with synaptic GABA receptors. Brain Research. 1978;148(4153-162. doi:10.1016/0006-8993(78)90385-2.
13. Johnston G a. R, Kennedy S M E, Lodge D. Muscimol Uptake, Release and Binding in Rat Brain Slices. Journal of Neurochemistry. 1978; 31(6):1519-1523. doi:10.1111/0471-4159.1978.tb06579.x.
14. Olpe H-R, Koella W P. The action of muscimol on neurones of the substantia nigra of the rat. Experientia. 1978; 34(2):235-235. doi:10.1007/BF01944700.
15. DeFeudis F V. Physiological and behavioral studies with muscimol. Neurochem Res. 1980; 5(10):1047-1068. doi:10.1007/BF00966163.
16. Krogsgaard-Larsen P, Frolund B, Frydenvang K. GABA uptake inhibitors. Design, molecular pharmacology and therapeutic aspects. Current Pharmaceutical Design. 2000; 6(12):1193-1209. doi:10.2174/1381612003399608.
17. Olsen R W. Drug Interactions at the Gaba Receptor-Ionophore Complex. Annual Review of Pharmacology and Toxicology. 1982; 22(1):245-277. doi:10.1146/annurev.pa.22.040182.001333.
18. Sieghart W. Structure, Pharmacology, and Function of GABAA Receptor Subtypes. In: Advances in Pharmacology. Vol 54. Academic Press; 2006:231-263. doi:10.1016/S1054-3589(06)54010-4.
19. Olsen R W, Chang C-SS, Li G, Hanchar H J, Wallner M. Fishing for allosteric sites on GABAA receptors. Biochemical Pharmacology. 2004; 68(8):1675-1684. doi:10.1016/j.bcp.2004.07.026.
20. Curtis D R, Lodge D, McLennan H. The excitation and depression of spinal neurones by ibotenic acid. The Journal of Physiology. 1979; 291(1):19-28. doi:10.1113/jphysio1.1979.sp012796.
21. Zinkand W C, Moore W C, Thompson C, Salama A I, Patel J. Ibotenic acid mediates neurotoxicity and phosphoinositide hydrolysis by independent receptor mechanisms. Molecular and Chemical Neuropathology. 1992; 16(1):1-10. doi:10.1007/BF03159956.
22. Nicotera P, Bellomo G, Orrenius S. Calcium-Mediated Mechanisms in Chemically Induced Cell Death. Annual Review of Pharmacology and Toxicology. 1992; 32(1):449-470. doi:10.1146/annurev.pa.32.040192.002313
23. Becker A, Grecksch G, Bernstein H-G, Hollt V, Bogerts B. Social behaviour in rats lesioned with ibotenic acid in the hippocampus: quantitative and qualitative analysis. Psychopharmacology. 1999; 144(4):333-338. doi:10.1007/s002130051015.
24. Isacson 0, Brundin P, Kelly P a. T, Gage F H, Bjorklund A. Functional neuronal replacement by grafted striatal neurones in the ibotenic acid-lesioned rat striatum. Nature. 1984; 311(5985):458-460. doi:10.1038/311458a0.
25. Heo H, Shin Y, Cho W, Choi Y, Kim H, Kwon Y K. Memory improvement in ibotenic acid induced model rats by extracts of Scutellaria baicalensis. Journal of Ethnopharmacology. 2009; 122(420-27. doi:10.1016/flep.2008.11.026.
26. Waser P G. The pharmacology of Amanita muscaria. Psychopharmacol Bull. 1967; 4(3):19-20.
27. Tamminga C A, Crayton J W, Chase T N. Muscimol: GABA agonist therapy in schizophrenia. The American Journal of Psychiatry. 1978; 135(6):746-747. doi:10.1176/ajp.135.6.746.
28. Krogsgaard-Larsen P, Falch E, Hjeds H. 2 Heterocyclic Analogues of GAB A: Chemistry, Molecular Pharmacology and Therapeutic Aspects. In: Ellis G P, West G B, eds. Progress in Medicinal Chemistry. Vol 22.; 1985:67-120. doi:10.1016/S0079-6468(08)70229-7.
29. Pahapill P A, Levy R, Dostrovsky J O, et al. Tremor arrest with thalamic microinjections of muscimol in patients with essential tremor. Annals of Neurology. 1999; 46(2):249-252.
30. Ludvig N, Baptiste S L, Tang H M, et al. Localized transmeningeal muscimol prevents neocortical seizures in rats and nonhuman primates: Therapeutic implications. Epilepsia. 2009; 50(4):678-693. doi:10.1111/0528-1167.2008.01914.x.
31. Tamminga C A, Crayton J W, Chase T N. Improvement in Tardive Dyskinesia After Muscimol Therapy. Arch Gen Psychiatry. 1979; 36(5):595-598. doi:10.1001/archpsyc.1979.01780050105013.
32. Cassady S L, Thaker G K, Moran M, Birt A, Tamminga C A. GABA agonist-induced changes in motor, oculomotor, and attention measures correlate in schizophrenics with tardive dyskinesia. Biological Psychiatry. 1992; 32(4):302-311. doi:10.1016/0006-3223(92)90035-X.
33. Chilton W S. Course of an intentional poisoning. Mcilvainea J Am Amat Mycol. Published online 1975. Accessed Jul. 1, 2020. https://agris.fao.ordagris-search/search.do?recordID=US201303019285.
34. Chapman A G. Glutamate and Epilepsy. J Nutr. 2000;130(4):1043S-1045S. doi:10.1093/jn/130.4.1043S.
35. Watanabe K, Tanaka T, Yonemasu Y. Ibotenic acid-induced limbic seizures and neuronal degeneration. Brain and Nerve (Japan). 1987; 39(6):505-508.
36. Kilbinger H. Presynaptic muscarine receptors modulating acetylcholine release. Trends in Pharmacological Sciences. 1984; 5:103-105. doi:10.1016/0165-6147(84)90383-3.
37. Mas A. Mushrooms, amatoxins and the liver. Journal of Hepatology. 2005; 42(2):166-169. doi:10.1016/j.jhep.2004.12.003.
38. Tracey A S, Crans D C, eds. Vanadium Compounds: Chemistry, Biochemistry, and Therapeutic Applications. American Chemical Society; Distributed by Oxford University Press; 1998.
39. Guedes da Silva M, Silva J, Frausto da Silva J. J-NJ Am Chem Soc. 1996; 118:7568-7573.
40. Nawi M, Riechel T L. The electrochemistry of amavadine, a vanadium natural product. Inorganica Chimica Acta. 1987; 136(1):33-39. doi:10.1016/S0020-1693(00)85559-0.
41. Smulyan H. The Beat Goes On: The Story of Five Ageless Cardiac Drugs. Am J Med Sci. 2018; 356(5):441-450. doi:10.1016/j.amjms.2018.04.011.
42. McLendon K, Preuss C V. Atropine. In: StatPearls. StatPearls Publishing; 2020. Accessed Jun. 30, 2020. http://www.ncbi.nlm.nih.gov/books/NBK470551/.
43. Tutone M, Lauria A, Almerico A M. Theoretical Determination of the pK a Values of Betalamic Acid Related to the Free Radical Scavenger Capacity: Comparison Between Empirical and Quantum Chemical Methods. Interdisciplinary Sciences: Computational Life Sciences. 2016; 8(2):177-185. doi:10.1007/s12539-015-0101-3
44. Kiho T, Katsurawaga M, Nagai K, Ukai S, Haga M. Structure and antitumor activity of a branched (13)-I3-d-glucan from the alkaline extract of Amanita muscaria. Carbohydrate Research. 1992; 224:237-243. doi:10.1016/0008-6215(92)84109-6.
45. Ruthes A C, Carbonero E R, Cordova M M, et al. Fucomannogalactan and glucan from mushroom Amanita muscaria: Structure and inflammatory pain inhibition. Carbohydrate Polymers. 2013; 98(1):761-769. doi:10.1016/j.carbpol.2013.06.061.
46. Hyoscyamine—DrugBank. Accessed Jun. 30, 2020. https://www.drugbank.ca/drugs/DB00424.
47. Girod P-A, Zryd J-P. Biogenesis of betalains: Purification and partial characterization of dopa 4,5-dioxygenase from Amanita muscaria. Phytochemistry. 1991; 30(1):169-174. doi:10.1016/0031-9422(91)84119-D.
48. Mueller L A, Hinz U, Zryd J-P. The formation of betalamic acid and muscaflavin by recombinant dopa-dioxygenase from Amanita. Phytochemistry. 1997; 44(4):567-569. doi:10.1016/S0031-9422(96)00625-5.
49. De Deurwaerdere P, Di Giovanni G, Millan M J. Expanding the repertoire of L-DOPA's actions: A comprehensive review of its functional neurochemistry. Progress in Neurobiology. 2017; 151:57-100. doi:10.1016/j.pneurobio.2016.07.002.
50. Fraser P J. Pharmacological actions of pure muscarine chloride. Br J Pharmacol Chemother. 1957; 12(1):47-52. Accessed Jun. 29, 2020. https://www.ncbi.nlm.nih.gov/pmc/articles/PMC1509643/.
51. Faulstich H, Buhring H J, Seitz J. Physical properties and function of phallolysin. Biochemistry. 1983; 22(19):4574-4580. doi:10.1021/bi002882035.
52. Lutsik-Kordovsky M, Stasyk T, Stoika R. Analysis of cytotoxicity of lectin and non-lectin proteins from Amanita mushrooms. Exp Oncol. 2001; 23:43-45.
53. Safer D J, Allen R P. The central effects of scopolamine in man. Biological Psychiatry. 1971; 3(4):347-355.
54. Hancianu M, Cioanca 0, Mihasan M, Hritcu L. Neuroprotective effects of inhaled lavender oil on scopolamine-induced dementia via anti-oxidative activities in rats. Phytomedicine. 2013; 20(5):446-452. doi:10.1016/j.phymed.2012.12.005.
55. Kaur H, Singh D, Singh B, Goel R K. Anti-amnesic effect of Ficus religiosa in scopolamine-induced anterograde and retrograde amnesia. Pharmaceutical Biology. 2010; 48(2):234-240. doi:10.3109/13880200903271306.
56. Bartus R T, Johnson H R. Short-term memory in the rhesus monkey: Disruption from the anti-cholinergic scopolamine. Pharmacology Biochemistry and Behavior. 1976; 5(1):39-46. doi:10.1016/0091-3057(76)90286-0.
57. Feeney K M. Revisiting Wasson's Soma: Exploring the effects of preparation on the chemistry of Amanita muscaria. Master's Thesis, Washington State University. Published online 2012. http://www.dissertations.wsu.edu/Thesis/Spring2012/k_feeney_013112. pdf.
58. Maggi A, Enna S J. Characteristics of muscimol accumulation in mouse brain after systemic administration. Neuropharmacology. 1979; 18(4):361-366. doi:10.1016/0028-3908(79)90143-6.

1. A composition comprising a first purified Amanita muscaria compound and a second purified Amanita muscaria compound each selected from the group consisting of: ibotenic acid, muscimol, muscazone, stizolobic acid, stizolobinic acid, muscarine, muscarufin, muscaflavin, betalamic acid, muscapurpurin, muscaaurins, acetylcholine, atropine, hyoscyamine, L-3,4-dihydroxyphenylalanine, phallolysin, scopolamine, fucomannogalactan, β(1→6)-branched (1→3)-β-d-glucan, amatoxins, phallotoxins, and the salts of these Amanita muscaria compounds.

2. A composition of claim 1, wherein the molar ratio of the first purified Amanita muscaria compound to the second purified Amanita muscaria compound in the composition is from about 0.1:100 to about 100:0.1, from about 1:100 to about 100:1, from about 1:50 to about 50:1, from about 1:25 to about 25:1, from about 1:20 to about 20:1, from about 1:10 to about 10:1, from about 1:5 to about 5:1, from about 1:2 to about 2:1 or may be about 1:1.

3. A formulation comprising the composition of claim 1 and an excipient.

4. A pharmaceutical formulation comprising the composition of claim 1 and a pharmaceutically acceptable excipient, wherein the first purified Amanita muscaria compound and the second purified Amanita muscaria compound are each present in a therapeutically effective amount.

5. A pharmaceutical composition of claim 4 further comprising a therapeutically effective amount of a serotonergic drug, a purified psilocybin derivative, a purified cannabinoid, or a purified terpene.

6. A pharmaceutical composition of claim 4, wherein the therapeutically effective amount of each of the first purified Amanita muscaria compound and the second purified Amanita muscaria compound separately ranges from about 0.5 mg-about 200 mg, about 1 mg-about 100 mg, about 2 mg-about 50 mg, about 5 mg-about 25 mg, or 25 mg.

7. A method of regulating the activity of a neurotransmitter receptor comprising the step of administering to a person in need thereof an effective dose of the composition of claim 1.

8. A method of regulating the activity of a neurotransmitter receptor comprising the step of administering to a person in need thereof the pharmaceutical formulation of claim 4.

9. A method of treating a psychological disorder, a compulsive disorder, or a depressive disorder comprising the step of administering to a person in need thereof an effective dose of the composition of claim 1.

10. A method of treating a psychological disorder, a compulsive disorder, or a depressive disorder comprising the step of administering to a person in need thereof the pharmaceutical formulation of claim 4.

11. A composition comprising a first purified Amanita muscaria compound, wherein the composition is substantially free of ibotenic acid.

12. The composition of claim 11, wherein the first purified Amanita muscaria compound comprises muscimol.

13. A composition comprising muscimol and ibotenic acid, wherein the composition exhibits a ratio of muscimol: ibotenic acid of greater than 5:1.

14. The composition of claim 13, wherein the composition exhibits a ratio of muscimol: ibotenic acid of 5:1 to 1000:1.

15. The composition of claim 13, wherein the composition exhibits a ratio of muscimol: ibotenic acid of 6:1 to 500:1.

16. The composition of claim 13, wherein the composition exhibits a ratio of muscimol: ibotenic acid of 10:1 to 100:1.

17. A composition comprising a first purified Amanita muscaria compound and a second purified Amanita muscaria compound, wherein the second purified Amanita muscaria compound comprises less than 10 wt. % of the composition.

18. The composition of claim 17, wherein the second purified Amanita muscaria compound comprises less than 5 wt. % of the composition.

19. The composition of claim 17, wherein the second purified Amanita muscaria compound comprises less than 1.0 wt. % of the composition.

20. The composition of claim 17, wherein the second purified Amanita muscaria compound comprises less than 0.5 wt. % of the composition.

21. The composition of claim 17, wherein the second purified Amanita muscaria compound comprises less than 0.25 wt. % of the composition.

22. The composition of claim 17, wherein the second purified Amanita muscaria compound comprises ibotenic acid.

23. The composition of claim 17, wherein the first purified Amanita muscaria compound comprises muscimol.

24. The composition of claim 17, wherein the composition comprises an Amanita Muscaria extract.
